(19) United States
(12) Patent Application Publication
(10) Pub. No.: US 2009/0047716 A1
(43) Pub. Date: Feb. 19, 2009

(54) REDUCTION PROCESSES FOR THE PREPARATION OF EZETIMIBE

(76) Inventors: Nurit PERLMAN, Kfar Saba (IL); Ayelet Fishman, Haifa, IL (US)

Correspondence Address: KENYON & KENYON LLP, ONE BROADWAY, NEW YORK, NY, 10004, US

(21) Appl. No.: 12/135,847

(22) Filed: Jun. 9, 2008

Related U.S. Application Data
(60) Provisional application No. 60/933,837, filed on Jun. 7, 2007.
(60) Provisional application No. 60/971,504, filed on Sep. 11, 2007.
(60) Provisional application No. 61/004,725, filed on Nov. 28, 2007.
(60) Provisional application No. 61/005,389, filed on Dec. 4, 2007.
(60) Provisional application No. 61/050,875, filed on May 6, 2008.

(51) Int. Cl. C12P 17/10; C07D 205/08

(52) U.S. Cl. 435/121; 540/200

(57) ABSTRACT

Processes for preparing ezetimibe-related compounds with a ketoreductase and for purifying ezetimibe are disclosed.

### CROSS-REFERENCE TO RELATED APPLICATIONS

This application claims the benefit of Provisional Application Ser. No. 60/933,837, filed Jun. 7, 2007, Provisional Application Ser. No. 60/971,504, filed Sep. 11, 2007; Provisional Application Ser. No. 61/004,725, filed Nov. 28, 2007, Provisional Application Ser. No. 61/005,389, filed Dec. 4, 2007, and Provisional Application Ser. No. 61/050,875, filed May 6, 2008, each of which is incorporated herein by reference in its entirety.

### FIELD OF THE INVENTION

The invention relates to reduction processes of ezetimibe intermediates to obtain ezetimibe or a derivative thereof.

### BACKGROUND OF THE INVENTION

Hydroxy-alkyl substituted azetidinones are useful as hypercholesterolemia agents in the treatment and prevention of atherosclerosis. Ezetimibe, 1-(4-fluorophenyl)-3(R)-[3-(4-fluorophenyl)-3(S)-hydroxypropyl]-4(S)-(4-hydroxyphenyl)-2-azetidinone, is a selective inhibitor of intestinal cholesterol and related phytosterol absorption. The empirical formula for ezetimibe is C₂₄H₂₁F₂NO₃, and its molecular weight is 409.4. Ezetimibe is a white, crystalline powder that is freely to very soluble in ethanol, methanol, and acetone and practically insoluble in water. Ezetimibe has the following chemical structure:

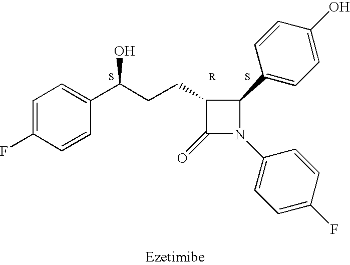

Ezetimibe is the active ingredient in the drug sold under the brand name ZETIA®, which is manufactured by Merck/Schering-Plough Pharmaceuticals. ZETIA® has been approved by the United States Food and Drug Administration for use in patients with high cholesterol to reduce low density lipoprotein (“LDL”) cholesterol and total cholesterol.

Ezetimibe can be prepared by reducing (3R,4S)-4-((4-benzyloxy)phenyl)-1-(4-fluorophenyl)-3-(3-(4-fluorophenyl)-3-oxopropyl)-2-azetidinone (“Compound 1”) with borane dimethyl sulfide complex or borane tetrahydrofuran complex in tetrahydrofuran in the presence of Corey's reagent and subsequently deprotecting the benzyl group, as shown in Scheme 1 below. The process is disclosed in U.S. Pat. Nos. 5,631,365 (“the '365 patent”) and 6,627,757, which are incorporated herein by reference. The starting material, Compound 1 or a similar compound, can be prepared by processes known in the art, for example, those disclosed in the '365 patent.

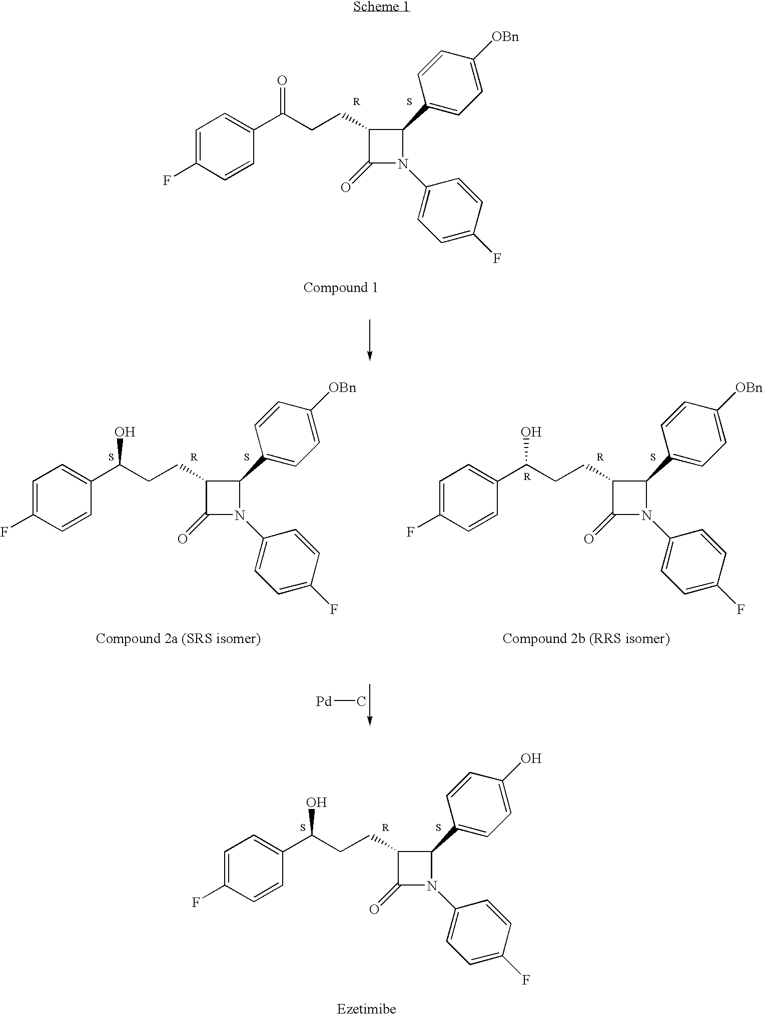

The reduction process produces two isomers, (3R,4S)-4-((4-benzyloxy)phenyl)-1-(4-fluorophenyl)-3-((S)-3-(4-fluorophenyl)-3-hydroxypropyl)-2-azetidinone (“Compound 2a”) and (3R,4S)-4-((4-benzyloxy)phenyl)-1-(4-fluorophenyl)-3-((R)-3-(4-fluorophenyl)-3-hydroxypropyl)-2-azetidinone (“Compound 2b”). Compound 2a is the desired isomer that produces ezetimibe of the proper chirality. Compound 2b is an undesirable isomer that is very difficult to remove both during reduction as well as the final synthesis to form ezetimibe. It has been reported that Compound 2b is typically produced in about 8 to 10% yield during the reduction process.

The '365 patent refers to the reduction of Compound 1 to Compound 2a by (R)-(+)-2-methyl-CBS-oxazaborolidine (“CBS”, Corey-Bakshi-Shibata catalyst) and borohydride dimethylsulfide complex (“BMS”), as illustrated below.

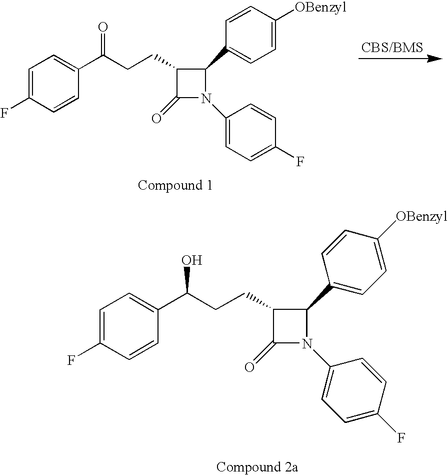

U.S. Pat. No. 6,133,001 refers to a process for stereoselective microbial reduction of ezetimibe-ketone to ezetimibe, as illustrated below.

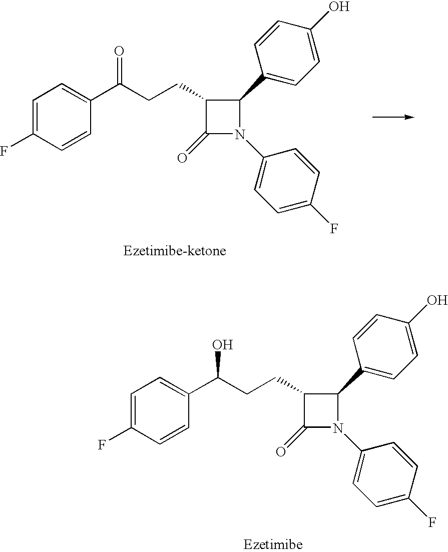

PCT publication No. WO 2005/066120 refers to an enantioselective reduction of ezetimibe-ketone to ezetimibe with (−)-B-chlorodiisopinocampheylborane (“DIP-Cl”).

There is a need for improved methods for preparing ezetimibe and derivatives thereof.

### SUMMARY OF THE INVENTION

In one embodiment, the invention encompasses a process for preparing a compound of formula I

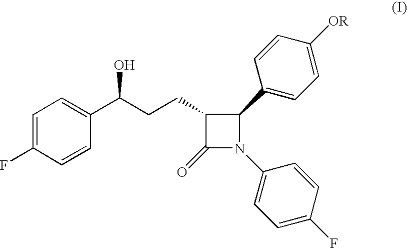

comprising combining a compound of formula II with an isolated, synthesized, or purified ketoreductase,

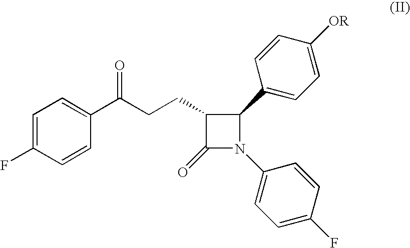

wherein R is H or a hydroxyl protecting group. Preferably, the ketoreductase is selected from the group consisting of the predominant enzyme in each of KRED-NADH-105, KRED-NADH-107, KRED-116, KRED-118, KRED-119, KRED-120, KRED-128, KRED-133, and mixtures thereof.

In one embodiment, the invention encompasses a process for preparing a compound of formula I

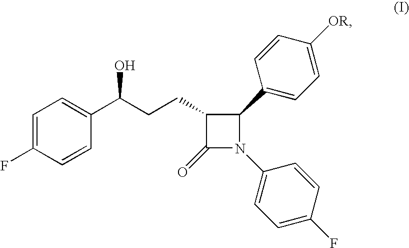

comprising combining a compound of formula II

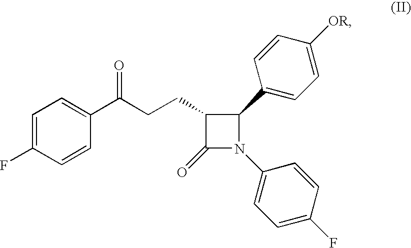

a ketoreductase enzyme, a co-factor, and a buffer having a pH of about 4 to about 8, wherein R is H or a hydroxyl protecting group.

In one embodiment, the invention encompasses a process for purifying ezetimibe from EZT-ketone comprising crystallizing ezetimibe from MIBK.

### DETAILED DESCRIPTION OF THE INVENTION

In one aspect, the present invention relates to a process for preparing a compound of formula I

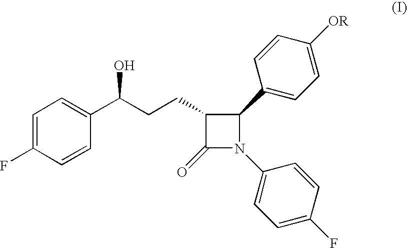

using enzymes as catalysts and optionally involves the use of water-miscible organic solvents.

As used herein, the term “yield” refers to percentage of a synthesized compound with respect to the original amount of starting material. It is determined by the area percentage under the peak of the synthesized compound relative to the total area in the HPLC chromatogram. Yield is typically measured during or immediately following a reaction.

As used herein, the term “purity” refers to the percentage of a compound with respect to other compounds. It is determined by the area percentage under the peak of the compound relative to the total area in the HPLC chromatogram. Purity is typically measured after a purification process.

As used herein, the term “d.e.” refers to diastereometric excess, defined as: (mole fraction of ezetimibe) minus (mole fraction of a R,R,S-diastereomer of ezetimibe).

$$d . e . = \frac{( \% Ezetimibe - \% R , R , S \text{-} diastereomer )}{( \% Ezetimibe + \% R , R , S \text{-} diastereomer ) .}$$

Ezetimibe has a S,R,S configuration, as shown below:

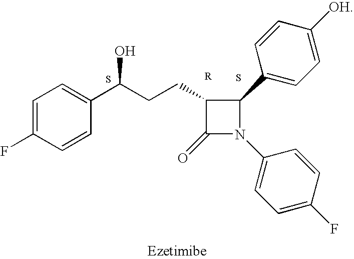

The R,R,S-diastereomer differs in that it has an (R) configuration at the chiral center on the third carbon in the propyl chain.

As used herein, the term “EZT” refers to ezetimibe, or 1-(4-fluorophenyl)-3(R)-[3-(4-fluorophenyl)-3(S)-hydroxypropyl]-4(S)-(4-hydroxyphenyl)-2-azetidinone. As used herein, the term “EZT RRS-isomer” refers to 1-(4-fluorophenyl)-3(R)-[3-(4-fluorophenyl)-3(R)-hydroxypropyl]-4(S)-(4-hydroxyphenyl)-2-azetidinone. As used herein, the term “ezetimibe-ketone” or “EZT-ketone” refers to 1-(4-fluorophenyl)-3(R)-[3-(4-fluorophenyl)-3-oxopropyl]-4(S)-(4-hydroxyphenyl)-2-azetidinone, having the following chemical structure:

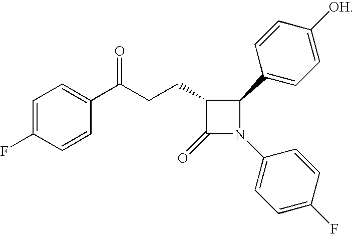

As used herein, the term “Compound 1” refers to (3R,4S)-4-((4-benzyloxy)phenyl)-1-(4-fluorophenyl)-3-(3-(4-fluorophenyl)-3-oxopropyl)-2-azetidinone; the term “Compound 2a” refers to (3R,4S)-4-((4-benzyloxy)phenyl)-1-(4-fluorophenyl)-3-((S)-3-(4-fluorophenyl)-3-hydroxypropyl)-2-azetidinone; and the term “Compound 2b” refers to (3R,4S)-4-((4-benzyloxy)phenyl)-1-(4-fluorophenyl)-3-((R)-3-(4-fluorophenyl)-3-hydroxypropyl)-2-azetidinone.

As used herein, when applied to an enzyme, the term “isolated” refers to an enzyme separated or removed from its native environment. Use of the term “isolated” indicates that a naturally occurring or recombinant enzyme has been removed from its normal cellular environment. Preferably, the isolated enzyme is in a cell-free system. The isolated enzyme can be crude or highly purified depending on the effort put in removing other materials. The term “isolated” does not imply that the enzyme is the only enzyme present, but that it is the predominant enzyme present (at least 10-20% more than any other enzyme). As used herein, when applied to an enzyme, the term “synthesized” refers to an enzyme that is prepared by chemical synthesis, recombinant means, or the combination thereof. As used herein, when applied to an enzyme, the term “purified” refers to an enzyme that is essentially free (at least about 90-95% pure) of non-enzymatic material or other enzymes.

As used herein, the term “dehydrogenase” or “dehydrogenase enzyme” refers to an enzyme that oxidizes a substrate by transferring one or more protons and a pair of electrons to an acceptor. Non-limiting examples of dehydrogenase include alcohol dehydrogenase, glucose dehydrogenase, formate dehydrogenase, and phosphite dehydrogenase. Glucose dehydrogenase (“GDH”) include, for example, those classified under the Enzyme Commission (“EC”) number of 1.1.1.47 and the Chemical Abstract Service (“CAS”) number 9028-53-9, and are commercially available, for example, from Codexis, Inc. (formerly BioCatalytics, Inc) under the catalog numbers GDH-102 to GDH-104. Formate dehydrogenase (“FDH”) include, for example, those classified under the EC number of 1.2.1.2 and the CAS number of 9028-85-7, and are commercially available, for example, from Codexis, Inc. under the catalog number FDH-101. Phosphite dehydrogenase (“PDH”) include, for example, those classified under the EC number of 1.20.1.1 and CAS number of 9031-35-0, and are commercially available, for example, from Codexis, Inc. under the catalog number PDH-101.

As used herein, the term “ketoreductase,” “ketoreductase enzyme,” or “KRED” refers to an enzyme that catalyzes the reduction of a ketone to form the corresponding alcohol. Ketoreductase enzymes include, for example, those classified under the EC numbers of 1.1.1. Such enzymes are given various names in addition to ketoreductase, including, but not limited to, alcohol dehydrogenase, carbonyl reductase, lactate dehydrogenase, hydroxyacid dehydrogenase, hydroxyisocaproate dehydrogenase, β-hydroxybutyrate dehydrogenase, steroid dehydrogenase, sorbitol dehydrogenase, aldoreductase, and the like. NADPH-dependent ketoreductases are classified under the EC number of 1.1.1.2 and the CAS number of 9028-12-0. NADH-dependent ketoreductases are classified under the EC number of 1.1.1.1 and the CAS number of 9031-72-5. Ketoreductases are commercially available, for example, from Codexis, Inc. under the catalog numbers KRED-100 to KRED-177.

As used herein, the term “co-factor” refers to a non-protein compound that operates in combination with an enzyme which catalyzes the reaction of interest. Co-factors include, for example, nicotinamide co-factors such as nicotinamide adenine dinucleotide (“NAD”), reduced nicotinamide adenine dinucleotide (“NADH”), nicotinamide adenine dinucleotide phosphate (“NADP⁺”), reduced nicotinamide adenine dinucleotide phosphate (“NADPH”), and any derivatives or analogs thereof.

As used herein, the term “MeOH” refers to methanol; the term “EtOAc” refers to ethyl acetate; the term “IPA” refers to isopropyl alcohol; the term “DMSO” refers to dimethyl sulfoxide”; the term “MIBK” refers to methyl isobutyl ketone; the term “DCM” refers to dichlormethane; the term “MTBE” refers to methyl tert-butyl ether, and the term “DTT” refers to dithiotreitol.

As used herein, the term “room temperature” or “RT” refers the ambient temperature of a typical laboratory, which is usually about 15° C. to about 30° C.

As used herein, the term “vacuum” refers to a pressure of about to 2 mmHg to about 100 mmHg.

In one embodiment, the invention encompasses a process for preparing a compound of formula I

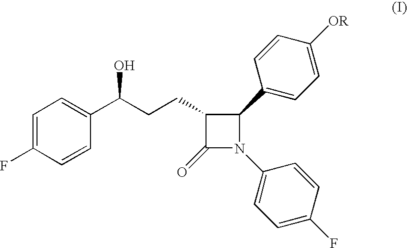

comprising combining a compound of formula II with an isolated, synthesized, or purified ketoreductase,

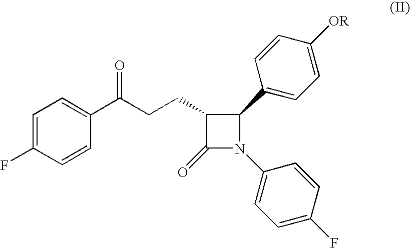

wherein R is H or a hydroxyl protecting group. Preferably, the ketoreductase is selected from the group consisting of the predominant enzyme in each of KRED-NADH-105, KRED-NADH-107, KRED-116, KRED-118, KRED-119, KRED-120, KRED-128, KRED-133, and mixtures thereof.

In one embodiment, the invention encompasses a process for preparing a compound of formula I

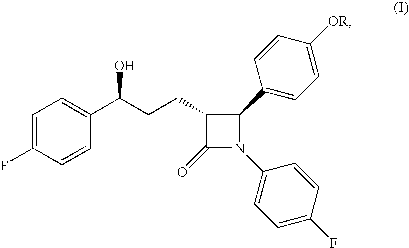

comprising combining a compound of formula II

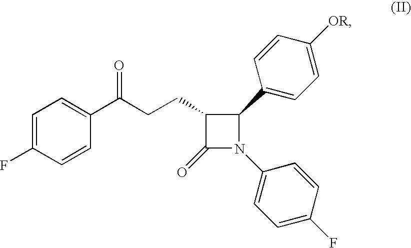

a biocatalyst, and a buffer, wherein R is H or a hydroxyl protecting group.

In one embodiment, the invention encompasses a process for preparing a compound of formula I

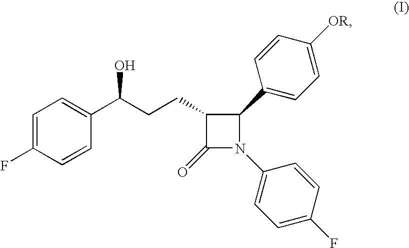

comprising combining a compound of formula II

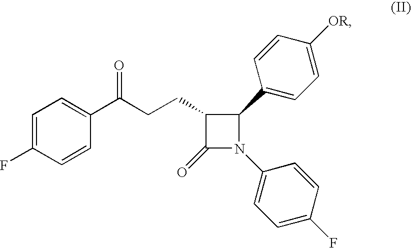

a ketoreductase enzyme, a co-factor, and a buffer having a pH of about 4 to about 8, wherein R is H or a hydroxyl protecting group.

When R is H, the compound of formula I is ezetimibe and the compound of formula II is ezetimibe-ketone. When R is a hydroxyl protecting group, the compound of formula II can be further deprotected to obtain ezetimibe. The deprotection can be done by known methods, for example those described in Example 6 of the '365 patent.

The reaction is an enzymatic process as illustrated below:

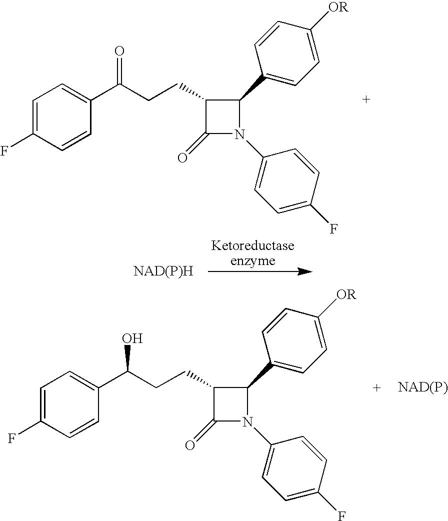

In this reaction, the ketoreductase stereoselectively reduces the carbonyl group. At the same time, a co-factor such as NADH or NADPH is oxidized.

Preferably, the hydroxyl protecting group is selected from the group consisting of benzyl, tert-butyloxycarbonyl, acyl, and silyl groups. Examples of suitable silyl protecting groups include, but are not limited to, (R^{a})(R^{b})(R^{c})—Si—, wherein R^{a}, R^{b} and R^{c} are each independently selected from the group consisting of C₁ to C₆ alkyl, phenyl, benzyl, acetyl, or the like.

Preferably, the ketoreductase is isolated. The ketoreductase can be separated from any host, such as mammals, filamentous fungi, yeasts, and bacteria. The isolation, purification, and characterization of a NADH-dependent ketoreductase is described in, for example, in Kosjek et al., Purification and Characterization of a Chemotolerant Alcohol Dehydrogenase Applicable to Coupled Redox Reactions, Biotechnology and Bioengineering, 86:55-62 (2004). Preferably, the ketoreductase is synthesized. The ketoreductase can be synthesized chemically or using recombinant means. The chemical and recombinant production of ketoreductases is described in, for example, in European patent no. EP 0918090. Preferably, the ketoreductase is synthesized using recombinant means in Escherichia coli. Preferably, the ketoreductase is purified, preferably with a purity of about 90% or more, more preferably with a purity of about 95% or more. Preferably, the ketoreductase is substantially cell-free.

Preferably, the ketoreductase is one that capable of producing ezetimibe with a d.e. of about 90% or higher in the processes of the invention. Preferably, the ketoreductase is one that capable of producing ezetimibe with a yield of about 50% or higher in the processes of the invention.

Preferably, the ketoreductase is selected from the group consisting of NADH-dependent ketoreductases and NADPH-dependent ketoreductases. Suitable ketoreductases include, but are not limited to, a) Codexis Inc's products with catalog numbers KRED-NADH-105, KRED-NADH-107, KRED-116, KRED-118, KRED-119, KRED-120, KRED-128, KRED-133, equivalent products thereof, and mixtures thereof; and b) the predominant enzyme in each of Codexis Inc's products with catalog numbers KRED-NADH-105, KRED-NADH-107, KRED-116, KRED-118, KRED-119, KRED-120, KRED-128, KRED-133, equivalent enzymes thereof, and mixtures thereof. As used herein, the term “equivalent” refers to an enzyme or product with similar or identical enzymatic activity. Preferably, the ketoreductase is selected from the group consisting of the predominant enzyme in each of Codexis Inc's products with catalog numbers KRED-NADH-105, KRED-NADH-107, KRED-116, KRED-118, KRED-119, KRED-120, KRED-128, KRED-133, and mixtures thereof. Preferably, the ketoreductase is selected from the group consisting the predominant enzyme in each of KRED-NADH-105, KRED-116, KRED-118, KRED-119, KRED-128, and mixtures thereof. More preferably, the ketoreductase is selected from the group consisting of the predominant enzyme in each of KRED-118, KRED-119, KRED-128, and mixtures thereof.

In one embodiment, the process of the invention further comprises combining a co-factor with the ketoreductase. Preferably, the co-factor is selected from the group consisting of NADH, NADPH, NAD⁺, NADP⁺, salts thereof, and mixtures thereof. Preferably, when the ketoreductase is NADH-dependent, the co-factor is selected from the group consisting of NADH, NAD⁺, salts thereof, and mixtures thereof. More preferably, the co-factor is NADH or a salt thereof. Preferably, when the ketoreductase is NADPH-dependent, the co-factor is selected from the group consisting of NADPH, NADP⁺, salts thereof, and mixtures thereof. More preferably, the co-factor is NADPH or a salt thereof.

In one embodiment, the process of the invention is carried out in a buffer. Preferably, the buffer has a pH of from about 4 to about 9, more preferably from about 4 to about 8, more preferably from about 5 to about 8, most preferably from about 6 to about 8 or about 5 to about 7. Preferably, the buffer is a solution of a salt. Preferably, the salt is selected from the group consisting of potassium phosphate, magnesium sulfate, and mixtures thereof. Optionally, the buffer comprises a thiol. Preferably, the thiol is DTT. Preferably, the thiol reduces the disulfide bond in the enzyme.

In one embodiment, the process of the invention is carried out at a temperature of about 10° C. to about 50° C. Preferably, the process is carried out at room temperature, at a temperature of about 24° C. to about 28° C., or at about 25° C. to about 35° C. Preferably, the process is carried out at a temperature of about 25° C. to about 30° C. Preferably, the process is carried out at a temperature of about 30° C.

Optionally, the reaction mixture further comprises a co-factor regeneration system. A co-factor regeneration system comprises a substrate and a dehydrogenase. The reaction between the substrate and dehydrogenase enzyme regenerates the co-factor. Preferably, the co-factor regeneration system comprises a substrate/dehydrogenase pair selected from the group consisting of D-glucose/glucose dehydrogenase, sodium formate/formate dehydrogenase, and phosphite/phosphite dehydrogenase. Preferably, the glucose dehydrogenase is selected from the group consisting of the predominant enzyme in each of Codexis Inc's products with catalog numbers GDH-102, GDH-103, GDH-104, and mixtures thereof. Preferably, the glucose dehydrogenase is the enzyme in GDH-104. Preferably, the formate dehydrogenase is the predominant enzyme in Codexis Inc's product with catalog number FDH-101. Preferably, the phosphite dehydrogenase is the predominant enzyme in Codexis Inc's product with catalog number PDH-101.

In one embodiment, the process of the invention further comprises adding a solvent. Preferably, the solvent is an organic solvent. Preferably, the organic solvent is water-miscible. Preferably the water-miscible organic solvent is selected from the group consisting of alcohols and DMSO. Preferably, the alcohol is a C₁-C₄ alcohol, more preferably methanol or IPA. The advantage of the preferred solvents used in this process, compared to the organic solvents used in prior art references, is that their medium is mostly water, which makes the reaction more environmentally friendly.

Preferably, the process comprises the following steps: (a) dissolving a compound of formula II in a solvent; and (b) combining the solution from (a) with a buffer containing a co-factor and a ketoreductase. Preferably, the obtained mixture is stirred for a period of time sufficient to obtain the compound of formula I. Preferably, the stirring is at a temperature of about 25° C. to about 35° C. or about 25° C. to about 40° C., more preferably at a temperature of about 24° C. to about 28° C., about 25° C. to about 30° C., or about 30° C. Preferably, the stirring is for about 0.5 hours or more, about 1.5 hours or more, or about 2.5 hours or more. Preferably, the stirring is for about 50 hours or less. Preferably, the stirring is for about 3 hours to about 40 hours, more preferably for about 6 hours to about 24 hours or about 6 hours to about 16 hours.

Optionally, a water-immiscible organic solvent is added to the reaction mixture, preferably after the stirring. Optionally, after the water-immiscible organic solvent is added, the reaction mixture is separated into an organic phase and an aqueous phase. Optionally, the compound of formula I is recovered by evaporating the organic phase. Examples of water-immiscible organic solvents include, but are not limited to, C₂-C₈ ethers, C₃-C₈ esters such as EtOAc, C₄-C₈ ketones such as MIBK, and halogenated hydrocarbons such as DCM. Preferably, the water-immiscible organic solvent is selected from the group consisting of EtOAc, MTBE, diethyl ether, and mixtures thereof. Preferably, the water-immiscible organic solvent is EtOAc.

Optionally, the reaction mixture, preferably after the stirring, is filtered to recover the solid product, which may optionally be further purified to obtain the compound of formula I.

Optionally, after the product is separated, the aqueous phase may be treated to recycle the enzyme, co-factor, and/or the dehydrogenase in the co-factor regeneration system. Optionally, the pH of the aqueous phase may be adjusted to obtain the desired pH. Optionally, the aqueous phase is evaporated to remove organic solvent residue. Optionally, the aqueous phase is reused in the process of the invention.

Preferably, the processes described above have high stereoselectivity toward ezetimibe. The d.e. of the ezetimibe obtained is preferably about 90% or higher, more preferably about 98% or higher, or about 99% or higher, and most preferably about 99.9% or higher.

Preferably, the processes described above has high yield. The yield of the compound of formula I obtained is preferably about 50% or higher, more preferably about 60% or higher, or about 70% or higher, or about 80% or higher and most preferably about 85% or higher.

In one embodiment, the invention encompasses a process for purifying ezetimibe from EZT-ketone comprising crystallizing ezetimibe from MIBK. Preferably, the process comprises: a) dissolving a sample comprising ezetimibe and EZT-ketone in MIBK; b) cooling the solution from step a); and c) recovering ezetimibe.

Preferably, step a) is performed under heating. Preferably, the heating is to a temperature of from about 50° C. to about reflux temperature, more preferably to about reflux temperature. Optionally, the cooling is to about room temperature or less, preferably to about 10° C. Optionally, a slurry is obtained after the cooling step. Optionally, ezetimibe is recovered from the slurry by filtering and optionally drying. The drying is preferably at about 40° C. to about 50° C., preferably under vacuum.

Preferably, the ezetimibe obtained has a purity of about 98% or more, more preferably about 99% or more.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

### HPLC Method for Determination of Impurity Profile of Ezetimibe

| Column & Packing: | Hypersil GOLD ™, C8, 150*4.6 mm, 3 μm, | | |
|---|---|---|---|
| | 25203-154630; | | |
| Buffer: | 0.05% TFA (trifluoracetic acid) | | |
| Eluent A; | 51:49, Methanol:Buffer | | |
| Eluent B: | 75:25, Acetonitrile:Eluent A | | |
| Gradient: | Time | % Eluent A | % Eluent B |
| | 0 | 100 | 0 |
| | 18 | 100 | 0 |
| | 35 | 0 | 100 |
| | 35.1 | 100 | 0 |

| | |
|---|---|
| Equilibrium time: | 7 min |
| Sample volume: | 5 μL |
| Flow Rate: | 1.5 mL/min |
| Detector: | 235 nm |
| Column temperature: | 30° C. |
| Autosampler temperature: | 10° C. |
| Diluent: | methanol |

### Typical Retention Times

| | Retention Time | |
|---|---|---|
| Compound | (min) | Relative Retention Time |
| Ezetimibe | ~18 | 1.0 |
| EZT RRS-isomer | ~20 | 1.10 |
| EZT-ketone | ~22 | ~1.25 |

### Example 1

### Reduction of EZT-Ketone with KRED-128

KRED-128 (5 mg, Codexis, Lot No. 090305CL, year of production: 2005) was dissolved in 5 ml buffer (containing 250 mM potassium phosphate, 0.5 mM DTT, 2 mM magnesium sulfate, 1.1 mM NADP⁺, 80 mM D-glucose, 10 U/ml glucose dehydrogenase, pH 7.0). A solution of EZT-ketone in MeOH (10 mg in 0.25 ml) was added. The mixture was stirred at 30° C. for 40 hours and monitored by HPLC. EtOAc (5 ml) was added and the phases were separated. The EZT in the organic phase was analyzed. (yield: 71.55%, d.e.: 99.9%).

### Example 2

### Reduction of EZT-Ketone with KRED-133

KRED-133 (5 mg, Codexis, Lot No. 113006MM, year of production: 2006) was dissolved in 5 ml buffer (containing 250 mM potassium phosphate, 0.5 mM DTT, 2 mM magnesium sulfate, 1.1 mM NADP⁺, 80 mM D-glucose, 10 U/ml glucose dehydrogenase, pH 7.0). A solution of EZT-ketone in MeOH (10 mg in 0.25 ml) was added. The mixture was stirred at 30° C. for 40 hours and monitored by HPLC. EtOAc (5 ml) was added and the phases were separated. The EZT in the organic phase was analyzed. (yield: 17.6%, d.e.: 99.3%).

### Example 3

### Reduction of EZT-Ketone with KRED-NADH-105

KRED-NADH-105 (5 mg, Codexis) was dissolved in 5 ml buffer (containing 250 mM potassium phosphate, 0.5 mM DTT, 2 mM magnesium sulfate, 1.3 mM NAD⁺, 80 mM D-glucose, 10 U/ml glucose dehydrogenase, pH 7.0). A solution of EZT-ketone in MeOH (20 mg in 0.25 ml) was added. The mixture was stirred at 30° C. over night and monitored by HPLC. EtOAc (5 ml) was added and the phases were separated. The EZT in the organic phase was analyzed. (yield: 57.11%, d.e.: 90.7%).

### Example 4

### Reduction of EZT-Ketone with KRED-NADH-107

KRED-NADH-107 (5 mg, Codexis) was dissolved in 5 ml buffer (containing 250 mM potassium phosphate, 0.5 mM DTT, 2 mM magnesium sulfate, 1.3 mM NAD⁺, 80 mM D-glucose, 10 U/ml glucose dehydrogenase, pH 7.0). A solution of EZT-ketone in MeOH (20 mg in 0.25 ml) was added and the mixture was stirred at 30° C. over night and monitored by HPLC. EtOAc (5 ml) was added and the phases were separated. The EZT in the organic phase was analyzed. (yield: 16.6%, d.e.: 98.5%).

### Example 5

### Reduction of EZT-Ketone with KRED-116

KRED-116 (5 mg, Codexis) was dissolved in 5 ml buffer (contains 250 mM potassium phosphate, 0.5 mM “DTT, 2 mM magnesium sulfate, 1.1 mM NADP⁺, 80 mM D-glucose, 10 U/ml glucose dehydrogenase, pH 7.0). A solution of EZT-ketone in MeOH (20 mg in 0.25 ml) was added and the mixture was stirred at 30° C. over night. EtOAc (5 ml) was added and the phases were separated. The organic phase was evaporated. The EZT in the residue was analyzed. (yield: 57.97%, d.e.: 99.9%).

### Example 6

### Reduction of EZT-Ketone with KRED-118

KRED-118 (5 mg, Codexis) was dissolved in 5 ml buffer (contains 250 mM potassium phosphate, 0.5 mM DTT, 2 mM magnesium sulfate, 1.1 mM NADP⁺, 80 mM D-glucose, 10 U/ml glucose dehydrogenase, pH 7.0). A solution of EZT-ketone in MeOH (20 mg in 0.25 ml) was added and the mixture was stirred at 30° C. over night. EtOAc (5 ml) was added and the phases were separated. The organic phase was evaporated. (yield: 87.59%, d.e.: 99.9%).

### Example 7

### Reduction of EZT-Ketone with KRED-119

KRED-119 (5 mg, Codexis, Lot No. 100407WW, year of production: 2007) was dissolved in 5 ml buffer (contains 250 mM potassium phosphate, 0.5 mM DTT, 2 mM magnesium sulfate, 1.1 mM NADP⁺, 80 mM D-glucose, 10 U/ml glucose dehydrogenase, pH 7.0). A solution of EZT-ketone in MeOH (20 mg in 0.25 ml) was added and the mixture was stirred at 30° C. over night. EtOAc (5 ml) was added and the phases were separated. The organic phase was evaporated. The EZT in the residue was analyzed. (yield: 83.19%, d.e.: 99.9%).

### Example 8

### Reduction of EZT-Ketone with KRED-120

KRED-120 (5 mg, Codexis) was dissolved in 5 ml buffer (contains 250 mM potassium phosphate, 0.5 mM DTT, 2 mM magnesium sulfate, 1.1 mM NADP⁺, 80 mM D-glucose, 10 U/ml glucose dehydrogenase, pH 7.0). A solution of EZT-ketone in MeOH (20 mg in 0.25 ml) was added and the mixture was stirred at 30° C. over night. EtOAc (5 ml) was added and the phases were separated. The organic phase was evaporated. The EZT in the residue was analyzed. (yield: 37.41%, d.e.: 99.9%).

### Example 9

### Reduction of EZT-Ketone with KRED-128

The solution of 10 mg EZT-ketone in 0.25 ml MeOH was added to the following solution: 5 mg KRED-128 (Codexis, Lot No. 090305CL, year of production: 2005), 36 mg (40 mM) D-glucose, 4 mg (1 mM) NADP⁺, 2.5 mg glucose dehydrogenase in 5 ml phosphate buffer (pH 7). The obtained milky mixture was stirred at 35° C. for 1.5 hours. The mixture was filtered, and the filtered material was analyzed (yield: 89.26%, d.e.: 99.9%).

### Example 10

### Reduction of EZT-Ketone with KRED-119

A solution of 600 mg EZT-ketone in 4 ml IPA was added to 20 ml 100 mM phosphate buffer solution (pH 6) containing: 800 mg (0.25M) D-glucose, 40 mg glucose dehydrogenase, 16 mg (1 mM) NADP⁺, and 20 mg KRED-119 (Codexis, Lot No. 100407WW, year of production: 2007. The obtained milky mixture was stirred at room temperature for 2.5 hours. (The reaction was monitored by HPLC until 74.4% conversion was observed). The mixture was filtered to obtain 0.59 g of wet product. (yield: 65.43%*). * The % conversion is lower than 74.4%, possibly because the reaction is reversible.

### Example 11

### Purification of Ezetimibe

2 g of mixture of ezetimibe and EZT-ketone (purity of ezetimibe: 82%; EZT-ketone content: 15.8%) was dissolved in 5 ml MIBK at reflux temperature. The solution was cooled to room temperature and stirred overnight. The obtained slurry was filtered and dried to obtain 0.9 g of white product (purity of ezetimibe: 99%).

What is claimed is:

1. A process for preparing a compound of formula I 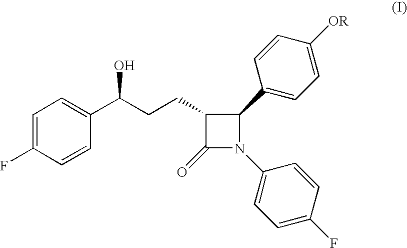
comprising combining a compound of formula II with an isolated, synthesized, or purified ketoreductase, 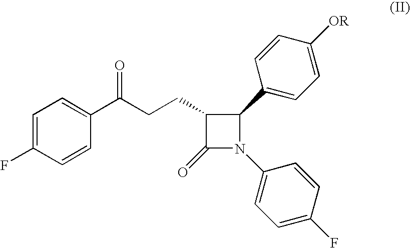
wherein R is H or a hydroxyl protecting group.

2. The process of claim 1, wherein the ketoreductase is isolated.

3. The process of any of claims 1-2, wherein the ketoreductase is synthesized.

4. The process of any of claims 1-3, wherein the ketoreductase is purified.

5. The process of any of claims 1-4, wherein R is hydrogen.

6. The process of any of claims 1-4, wherein R is a hydroxyl protecting group.

7. The process of claim 6, wherein R is a selected from the group consisting of benzyl, tert-butyloxycarbonyl, acyl, and silyl groups.

8. The process of claim 7, wherein the silyl group is (R^{a})(R^{b})(R^{c})—Si—, wherein R^{a}, R^{b} and R^{c} are each independently selected from the group consisting of C₁ to C₆ alkyl, phenyl, acetyl, and benzyl groups.

9. The process of any of claims 1-8, wherein the ketoreductase is selected from the group consisting of the predominant enzyme in each of KRED-NADH-105, KRED-NADH-107, KRED-116, KRED-118, KRED-119, KRED-120, KRED-128, KRED-133, and mixtures thereof

10. The process of any of claim 9, wherein the ketoreductase is selected from the group consisting of the predominant enzyme in each of KRED-NADH-105, KRED-116, KRED-118, KRED-119, KRED-128, and mixtures thereof.

11. The process of claim 10, wherein the ketoreductase is selected from the group consisting of the predominant enzyme in each of KRED-118, KRED-119, KRED-128, and mixtures thereof.

12. The process of any of claims 1-11, further comprises combining a co-factor with the ketoreductase, wherein the co-factor is selected from the group consisting of NADH, NADPH, NAD⁺,NADP⁺, salts thereof, and mixtures thereof.

13. The process of claim 12, wherein the co-factor is NADH or a salt thereof.

14. The process of claim 12, wherein the co-factor is NADPH or a salt thereof.

15. The process of any of claims 1-14, wherein the process is carried out in a buffer having a pH of about 4 to about 9.

16. The process of claim 15, wherein the buffer has a pH of about 4 to about 8.

17. The process of claim 16, wherein the buffer has a pH of about 6 to about 8.

18. The process of any of claims 15-17, wherein the buffer is a solution of at least one salt selected from the group consisting of potassium phosphate, magnesium sulfate.

19. The process of any of claims 15-18, wherein the buffer comprises dithiotreitol.

20. The process of any of claims 1-19, wherein the process is carried out at a temperature of about 10° C. to about 50° C.

21. The process of claim 20, wherein the process is carried out at a temperature of about 25° C. to about 35° C.

22. The process of claim 21, wherein the process is carried out at a temperature of about 25° C. to about 30° C.

23. The process of any of claims 1-22, wherein the reaction mixture further comprises a co-factor regeneration system.

24. The process of claim 23, wherein the co-factor regeneration system comprises a substrate/dehydrogenase pair selected from the group consisting of D-glucose/glucose dehydrogenase, sodium formate/formate dehydrogenase, and phosphite/phosphite dehydrogenase.

25. The process of claim 24, wherein the substrate/dehydrogenase pair is D-glucose/glucose dehydrogenase.

26. The process of claim 25, wherein the glucose dehydrogenase is selected from the group consisting of the predominant enzyme in each of GDH-102, GDH-103, GDH-104, and mixtures thereof.

27. The process of claim 26, wherein the glucose dehydrogenase is the enzyme in GDH-104.

28. The process of claim 24, wherein the substrate/dehydrogenase pair is sodium formate/formate dehydrogenase.

29. The process of claim 28, wherein the formate dehydrogenase is the predominant enzyme in FDH-101.

30. The process of claim 24, wherein the substrate/dehydrogenase pair is sodium phosphite/phosphite dehydrogenase.

31. The process of claim 30, wherein the phosphite dehydrogenase is the predominant enzyme in PDH-101.

32. The process of any of claims 1-31, further comprising adding a solvent.

33. The process of claim 32, wherein the solvent is a water-miscible organic solvent.

34. The process of claim 33, wherein the solvent is selected from the group consisting of alcohols and dimethyl sulfoxide.

35. The process of claim 34, wherein the alcohol is a C₁-C₄ alcohol.

36. The process of claim 35, wherein the alcohol is methanol or isopropyl alcohol.

37. The process of any of claims 32-36, comprising:
(a) dissolving the compound of formula II in a solvent;
(b) combining the solution from (a) with a buffer containing a co-factor and a ketoreductase.

38. The process of any of claims 1-37, wherein the reaction mixture is stirred for about 3 hours to about 40 hours.

39. The process of claim 38, wherein the reaction mixture is stirred for about 14 hours to about 24 hours.

40. The process of any of claims 38-39, wherein the reaction mixture is stirred at a temperature of about 25° C. to about 35° C.

41. The process of any of claims 1-40, further comprising recovering the product by filtering the reaction mixture.

42. The process of any of claims 1-41, wherein a water immiscible organic solvent is added to the reaction mixture

43. The process of claim 42, wherein the water immiscible organic solvent is added to the reaction mixture after stirring.

44. The process of any of claims 42-43, wherein the reaction mixture is separated into an organic phase and an aqueous phase after the water immiscible organic solvent is added.

45. The process of any of claims 42-44, wherein the water immiscible organic solvent is selected from the group consisting of C₂-C₈ ethers, C₃-C₈ esters, C₄-C₈ ketones, halogenated hydrocarbons, and mixtures thereof.

46. The process of any of claims 44-45, further comprising recovering the product by evaporating the organic phase.

47. The process of any of claims 1-46, wherein the yield of the compound of formula I obtained is about 50% or higher.

48. The process of claim 47, wherein the yield is about 60% or higher.

49. The process of claim 48, wherein the yield is about 70% or higher.

50. The process of claim 49, wherein the yield is about 80% or higher.

51. The process of claim 50, wherein the yield is about 85% or higher.

52. The process of any of claims 1-5 and 9-51, wherein the compound of formula I is ezetimibe.

53. The process of claim 52, wherein the diastereometric excess of ezetimibe is about 90% or higher.

54. The process of claim 53, wherein the diastereometric excess of ezetimibe is about 98% or higher.

55. The process of claim 54, wherein the diastereometric excess of ezetimibe is about 99% or higher.

56. The process of claim 55, wherein the diastereometric excess of ezetimibe is about 99.9% or higher.

57. The ezetimibe produced by the process of any of claims 1-56,

58. A process for purifying ezetimibe from EZT-ketone crystallizing ezetimibe from methyl isobutyl ketone.

59. The process of claim 58, comprising:
a) dissolving a sample comprising ezetimibe and EZT-ketone in methyl isobutyl ketone;
b) cooling the solution from step a); and
c) recovering ezetimibe.

60. The process of claim 59, wherein step a) is performed under heating.

61. The process of claim 60, wherein the heating is to a temperature of from about 50° C. to about reflux temperature.

62. The process of claim 61, wherein the heating is to about reflux temperature.

63. The process of any of claims 59-62, wherein the cooling is to about room temperature or less.

64. The process of claim 63, wherein the cooling is to about 10° C.

65. The process of any of claims 59-64, wherein a slurry is obtained after the cooling step, and wherein ezetimibe is recovered from the slurry by filtering.

66. The process of any of claims 58-65, wherein the obtained ezetimibe has a purity of about 98% or more.

67. The process of claim 66, wherein the obtained ezetimibe has a purity of about 99% or more.

68. The ezetimibe obtained by the process of any of claims 58-67.
